(19) United States
(12) Patent Application Publication
(10) Pub. No.: US 2026/0258345 A1
(43) Pub. Date: Sep. 3, 2026

(54) LIMOSILACTOBACILLUS REUTERI STRAIN AND IMMUNITY ENHANCEMENT USES THEREOF

(71) Applicant: KoBioLabs, Inc., Gwanak-gu, Seoul (KR)

(72) Inventors: Gwang Pyo KO, Seoul (KR); Cheonghoon LEE, Seoul (KR); Doseon CHOI, Seoul (KR); Hyun Ju YOU, Incheon (KR); Bo-Ram CHO, Seoul (KR); Sung Jae JANG, Seoul (KR)

(21) Appl. No.: 19/156,098

(22) PCT Filed: Feb. 14, 2024
(86) PCT No.: PCT/KR2024/002109

§ 371 Date: Aug. 13, 2025

(30) Foreign Application Priority Data
Feb. 15, 2023 (KR) 10-2023-0020133

(51) Int. Cl. C12N 1/205; A61K 35/00; A61P 29/00; A61P 31/16; A61P 37/04; C12R 1/01

(52) U.S. Cl. CPC C12N 1/205; A61P 29/00; A61P 31/16; A61P 37/04; A61K 2035/115; C12R 2001/01

(57) ABSTRACT

The present application relates to a Limosilactobacillus reuteri strain and immunity enhancement uses thereof.

### TECHNICAL FIELD

The present disclosure relates to a Limosilactobacillus reuteri strain and a use for enhancing immunity thereof.

### BACKGROUND ART

Immunity refers to a defensive mechanism in which the internal environment of a living organism recognizes various substances that have invaded from the outside as foreign materials and eliminates or metabolizes them. However, immune function disorder may occur due to various causes, and various immune-enhancing agents are required to prevent or treat them.

The gut microbiota residing in the human gastrointestinal tract provides various health benefits through close interaction with the human host. Recently, the demand for probiotics has increased significantly worldwide, and there is a need to develop health functional foods and drugs using probiotics.

### DISCLOSURE

### Technical Problem

Under these circumstances, the present inventors have identified that a Limosilactobacillus reuteri KBL346 strain exhibits an immune-enhancing effect by activating immune cells. Accordingly, an embodiment of the present application is directed to providing of a use of the Limosilactobacillus reuteri KBL346 strain for enhancing immunity, which can be used for the prevention or treatment of immunosuppression.

In addition, the present inventors have found that the Limosilactobacillus reuteri KBL346 strain has an effect of suppressing weight loss caused by influenza virus, lowering mortality rate, and improving pulmonary inflammation. Accordingly, another embodiment of the present application is to provide anti-influenza virus activity and immune-enhancing efficacy of the Limosilactobacillus reuteri KBL346 strain, which can be used in the prevention and improvement of infectious respiratory immune diseases including influenza virus infection in the future.

### Technical Solution

An embodiment of the present application relates to a Limosilactobacillus reuteri KBL346 strain deposited under Accession No. KCTC 15268BP, a use of the strain for enhancing immunity, a use of the strain for preventing or treating immunosuppression, a use of the strain for anti-influenza virus, or a use of the strain for preventing or treating influenza virus infection. The strain may have a 16S rRNA sequence set forth in SEQ ID NO: 3.

Hereinafter, the present disclosure will be described in further detail.

An embodiment of the present application relates to a composition for enhancing immunity comprising at least one selected from the group consisting of a Limosilactobacillus reuteri KBL346 strain deposited under Accession No. KCTC 15268BP, a culture of the strain, a lysate of the strain, and an extract of the strain. In Example of the present application, when a strain according to an embodiment of the present application was treated on macrophages, it activated the macrophages. Accordingly, the immune enhancement may be activating macrophages, and the composition may be an immune stimulator. The strain may specifically be a Limosilactobacillus reuteri subsp. reuteri strain.

Specifically, the composition may have one or more characteristics selected from the group consisting of the following (1) to (10):
(1) promotion of phagocytic activity of macrophages, for example, a phagocytic activity of macrophages is 1.1-fold or higher, 1.2-fold or higher, 1.3-fold or higher, 1.4-fold or higher, or 1.45-fold or higher, compared to an untreated control group;
(2) promotion of nitric oxide (NO) production by macrophages, for example, a nitric oxide production level of the macrophages is 1.5-fold or higher, 2-fold or higher, 2.5-fold or higher, 3-fold or higher, 4-fold or higher, 5-fold or higher, 6-fold or higher, 7-fold or higher, 8-fold or higher, 8.5-fold or higher, or 9-fold or higher, compared to an untreated control group;
(3) promotion of cyclooxygenase-2 (COX-2) expression in macrophages;
(4) promotion of prostaglandin E2 (PGE2) secretion by macrophages, for example, a secretion amount of PGE2 of macrophages is 2-fold or higher, 3-fold or higher, 4-fold or higher, 5-fold or higher, 8-fold or higher, 10-fold or higher, 15-fold or higher, 20-fold or higher, 22-fold or higher, 25-fold or higher, 26-fold or higher, 27-fold or higher, 28-fold or higher, 29-fold or higher, or 30-fold higher, compared to an untreated control group;
(5) promotion of cytokine secretion by macrophages, for example, promotion of tumor necrosis factor-α (TNF-α) and/or interleukin-6 (IL-6) secretion;
(6) promotion of expression and phosphorylation of nuclear factor kappa B (NF-κB) and/or nuclear factor of kappa light polypeptide gene enhancer in B-cells inhibitor alpha (IκBα) in macrophages;
(7) promotion of activating mitogen-activated protein kinases (MAPKs) of macrophages;
(8) promotion of recovery from viral infection, for example, promotion of recovery from influenza virus infection;
(9) prevention of weight loss caused by immunosuppression; and
(10) increase in monocytes in blood.

Another embodiment of the present application relates to a food composition comprising the composition for enhancing immunity. The food may be a health functional food, and may be in the form of, for example, meat products, bakery, chocolates, candies, jelly, snacks, confectionery, kimchi, fermented sauces, cheese, dairy products, powders, beverages, or vitamin complexes.

Another embodiment of the present application relates to a quasi-drug composition comprising the composition for enhancing immunity. The quasi-drug composition may be in a form selected from the group consisting of toothpaste, mouthwash, oral spray, oral ointment, oral cleanser, oral deodorizer, bandage, and pest (mosquito, tick, and the like) repellent.

Another embodiment of the present application relates to a composition for preventing or treating immunosuppression, comprising at least one selected from the group consisting of the Limosilactobacillus reuteri KBL346 strain deposited under Accession No. KCTC 15268BP, a culture of the strain, a lysate of the strain, and an extract of the strain.

The immunosuppression is caused by immune cells failing to defend against external pathogens or cancer cells due to immune deficiency, and may be, for example, immunodeficiency syndromes, and specifically, secondary or acquired immunodeficiency.

The immunosuppression may be one or more selected from the group consisting of herpes zoster, tuberculosis, meningitis, bronchiolitis, and bacterial or viral infections. The viral infection may be, for example, an influenza virus infection.

The influenza virus is the major causative agent of seasonal influenza epidemics, commonly referred to as “flu,” and is known to be highly contagious compared to other respiratory viruses, so as to produce approximately 5 million severe cases annually due to influenza virus infection. Influenza virus infection is accompanied by symptoms such as fever, cough, runny nose, and phlegm, can cause complications in those with weak immunity, and has a high mortality rate, so caution is required.

The influenza virus invades the mucosa of the upper respiratory tract and causes respiratory diseases. Virus particles released from the respiratory tract of an infected patient through coughing or sneezing are transmitted to others via the respiratory tract, thereby affecting the lungs and airways. In contrast, norovirus infection occurs through the consumption of contaminated food or beverages. The virus infects small intestinal cells, exhibiting a completely different infection route from that of the influenza virus. Accordingly, the antiviral effect against norovirus known in the prior art is typically exerted by a mechanism that physically interferes with epithelial cells in the small intestine or the norovirus itself to inhibit infection. However, in the case of the influenza virus, antiviral efficacy based on physical contact is not expected. Therefore, due to the difference in mechanism of action, it is difficult to predict whether an agent exhibiting antiviral activity against norovirus would also exhibit antiviral activity against the influenza virus.

Another embodiment of the present application relates to a composition for anti-influenza virus, comprising at least one selected from the group consisting of the Limosilactobacillus reuteri KBL346 strain deposited under Accession No. KCTC 15268BP, a culture of the strain, a lysate of the strain, and an extract of the strain. In Example of the present application, when the treatment of the strain according to the present embodiment to an influenza virus infection model, it reduced significantly the severity and mortality of influenza virus infection.

Specifically, the composition may exhibit at least one characteristic selected from the group consisting of the following (1) to (4):
(1) Prevention of body weight loss caused by influenza virus infection, for example, the body weight after infection of 75% or higher, 79% or higher, 80% or higher, 81% or higher, 82% or higher, 82.5% or higher, 84% or higher, 85% or higher, 90% or higher, 91% or higher, 92% or higher, 93% or higher, or 94% or higher, based on 100% of the body weight before influenza virus infection (wherein the body weight after infection may be measured on day 7 after influenza virus infection);
(2) Reduction in mortality rate caused by influenza virus infection, for example, a survival rate of 10% or higher, 20% or higher, 30% or higher, 40% or higher, 50% or higher, 55% or higher, 60% or higher, 65% or higher, 70% or higher, 75% or higher, 80% or higher, 85% or higher, or 90% or higher (wherein the survival rate may be measured 21 days after influenza virus infection);
(3) Alleviation of pulmonary inflammation; and
(4) Reduction in serum immunoglobulin G (IgG) levels, for example, a serum lgG concentration of 0.9-fold or less, 0.8-fold or less, 0.7-fold or less, or 0.65-fold or less compared to an untreated control.

Another embodiment of the present application relates to a composition for preventing or treating influenza virus infection, comprising at least one selected from the group consisting of the Limosilactobacillus reuteri KBL346 strain deposited under Accession No. KCTC 15268BP, a culture of the strain, a lysate of the strain, and an extract of the strain.

The influenza virus infection may be at least one selected from the group consisting of flu, pneumonia, Reye's syndrome, acute respiratory failure, myocarditis, bronchitis, otitis media, pharyngitis, sinusitis, empyema, Guillain-Barre syndrome, and encephalitis.

The symptoms of the influenza virus infection may be one or more selected from the group consisting of fever, chills, nausea, myalgia, fatigue, cough, dyspnea, shortness of breath, pneumonia, sputum production, sore throat, headache, hemoptysis, and diarrhea.

In the present application, the term “strain” may refer to a live bacterium or heat-inactivated bacterium according to an embodiment of the present application.

In the present application, the term “culture of a strain” means a product obtained by culturing a strain according to an embodiment of the present application, and the culture may be a whole culture of the strain according to an embodiment of the present application, or a dilution, a concentrate, a dried product, a lyophilized product, a lysate, and/or a fraction thereof. The concentrate may be obtained by centrifuging or evaporating the culture, the dried product may be obtained by drying the culture using a dryer or the like, the lyophilized product may be obtained by lyophilizing the culture using a lyophilizer or the like, the lysates may be obtained by physically or ultrasonically treating the strain or the culture, and the fraction may be obtained by subjecting the culture, the lysate, and the like to a method such as centrifugation, chromatography, or the like. The culture may be in a solid phase (solid, for example, a dried product), a liquid phase (liquid), or a fluid phase, but is not limited thereto. In an example, the culture may mean the whole medium including the cultured strains, their metabolites, and/or extra nutrients, obtained by culturing the strain according to an embodiment of the present application for a certain period of time. In an example, the culture may be one in which the strain according to an embodiment of the present application is removed or not removed. In an example, the culture may mean the remaining components excluding the strain (bacteria) in the culture which is obtained by culturing the strain according to an embodiment of the present application in a medium. In an example, the culture may be a culture solution (or culture product) obtained by culturing the strain according to an embodiment of the present application in a medium from which the strains (bacteria) are removed. The culture solution (or culture product) from which the strains are removed may be a cell-free culture solution (or culture product) or a culture solution containing dead cells, and may be, for example, a filtrate (centrifuged supernatant) obtained by removing the strains through filtration or centrifugation, and/or a culture solution (or dried product of the culture solution) containing dead cells. Specifically, the culture may exhibit anti-inflammatory activity at a level equivalent to that exhibited by the strain according to an embodiment of the present application, or activity for preventing, improving or treating an inflammatory disease.

In the present application, the term “lysate of strain” may mean a product obtained by performing lysis a strain according to an embodiment of the present application by chemical or physical force. Specifically, the lysate may exhibit anti-inflammatory activity at a level equivalent to that exhibited by the strain according to an embodiment of the present application, or activity for preventing, improving, or treating inflammatory diseases.

The term “extract” in the present application may mean a product obtained by extracting a strain according to an embodiment of the present application, a culture of the strain, a lysate of the strain, or a mixture thereof, regardless of the extraction method, extraction solvent, extracted component, or form of the extract, and is a broad concept including all materials that can be obtained by processing or handling by another method after extraction. For example, the extract may be an extract of the strain according to an embodiment of the present application, an extract of the culture of the strain, or an extract of the lysate of the strain. Specifically, the extract may exhibit anti-inflammatory activity, or activity for preventing, improving, or treating inflammatory diseases, at a level equivalent to the activity exhibited by the strain according to an embodiment of the present application, a culture of the strain, or a lysate of the strain.

The composition according to an embodiment of the present invention may include Limosilactobacillus reuteri KBL346 strain deposited under Accession No. KCTC 15268BP at a concentration of 1.0×10³ to 1.0×10¹⁵ CFU, 1.0×10³ to 1.0×10¹⁴ CFU, 1.0×10³ to 1.0×10¹³ CFU, 1.0×10³ to 1.0×10¹² CFU, 1.0×10³ to 1.0×10¹¹ CFU, 1.0×10³ to 1.0×10¹⁰ CFU, 1.0×10⁵ to 1.0×10¹⁵ CFU, 1.0×10⁵ to 1.0×10¹⁴ CFU, 1.0×10⁵ to 1.0×10¹³ CFU, 1.0×10⁵ to 1.0×10¹² CFU, 1.0×10⁵ to 1.0×10¹¹ CFU, 1.0×10⁵ to 1.0×10¹⁰ CFU, 1.0×10⁷ to 1.0×10¹⁵ CFU, 1.0×10⁷ to 1.0×10¹⁴ CFU, 1.0×10⁷ to 1.0×10¹³ CFU, 1.0×10⁷ to 1.0×10¹² CFU, 1.0×10⁷ to 1.0×10¹¹ CFU, 1.0×10⁷ to 1.0×10¹⁰ CFU, 1.0×10⁸ to 1.0×10¹⁵ CFU, 1.0×10⁸ to 1.0×10¹⁴ CFU, 1.0×10⁸ to 1.0×10¹³ CFU, 1.0×10⁸ to 1.0×10¹² CFU, 1.0×10⁸ to 1.0×10¹¹ CFU, or 1.0×10⁸ to 1.0×10¹⁰ CFU.

In the present application, the term ‘prevention’ means suppressing or delaying the onset of a disease, disorder, or condition. If the onset of a disease, disorder, or condition is suppressed or delayed for a predetermined period of time, prevention may be considered complete.

In the present application, the term ‘treatment’ means partially or completely alleviating, improving, alleviating, inhibiting, or delaying a specific disease, disorder, and/or condition or symptom, reducing the severity, or reducing the occurrence of one or more symptoms or characteristics.

The composition of the present application, for example, a pharmaceutical composition or a food composition, may additionally comprise one or more active ingredients exhibiting the same or similar function in addition to the above-mentioned active ingredients.

In addition, the composition according to the present application, for example, a pharmaceutical composition or a food composition, can be manufactured in a unit dosage form or manufactured by introducing into a multi-dose container by formulating using a pharmaceutically acceptable carrier according to a method that can be clearly performed by a person having ordinary skill in the art to which the present invention pertains. The term ‘carrier’ in the present application means a compound that facilitates the addition of a compound into cells or tissues, and the term ‘pharmaceutically acceptable’ means a composition that is physiologically acceptable and does not typically cause an allergic reaction such as gastrointestinal disorder or dizziness or a similar reaction when administered to a human.

The pharmaceutically acceptable carriers mentioned above are those commonly used in the preparation of formulations and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but are not limited to.

In addition, the composition according to the present application, for example, a pharmaceutical composition or a food composition, may additionally comprise additives such as a filler, an anticoagulant, a lubricant, a wetting agent, a flavoring agent, an emulsifier, a preservative, etc., in addition to the above components. In the present application, the content of the additives included in the composition is not particularly limited and may be appropriately adjusted within the content range used in conventional formulations.

In addition, the composition according to the present application, for example, a pharmaceutical composition or a food composition, may be formulated as an oral preparation. Non-limiting examples of the oral preparation include tablets, troches, lozenges, aqueous suspensions, oily suspensions, prepared powders, granules, emulsions, hard capsules, soft capsules, syrups, or elixirs. In order to formulate a pharmaceutical composition or a food composition according to the present application for oral administration, a binder such as lactose, saccharose, sorbitol, mannitol, starch, amylopectin, cellulose or gelatin; an excipient such as dicalcium phosphate; a disintegrant such as corn starch or sweet potato starch; magnesium stearate, calcium stearate, sodium stearyl fumarate, and the like; and a sweetener, a flavoring agent, a syrup, and the like can also be used. Furthermore, in the case of capsules, in addition to the above-mentioned substances, a liquid carrier such as fatty oil can be additionally used.

In the present application, the term ‘excipient’ means a substance other than a therapeutic agent and means a carrier or medium used for delivering a therapeutic agent or added to a pharmaceutical composition. This improves handling and storage characteristics or allows and facilitates the formation of a unit dosage of the composition.

The composition according to the present application, for example, the pharmaceutical composition, may be formulated and used in various forms, such as oral dosage forms such as liquids, suspensions, powders, granules, tablets, capsules, pills, extracts, emulsions, syrups, aerosols, injections of sterile injectable solutions, and the like, according to a conventional method according to each intended use, and may be administered orally or through various routes including intravenous, intraperitoneal, subcutaneous, rectal, and topical administration. The term ‘oral administration’ in this application means a substance prepared so that an active substance can be digested, i.e., administered to the gastrointestinal tract for absorption.

The preferred dosage of a composition according to the present application, for example, a pharmaceutical composition or a food composition, may vary depending on the patient's condition and weight, age, sex, health condition, dietary constitution, nature of the preparation, degree of disease, administration time of the composition, administration method, administration period or interval, excretion rate, and drug form, and may be appropriately selected by a person skilled in the art.

The term ‘effective dosage’ in this application means the amount of a composition of an active ingredient sufficient to treat a specific symptom. This may vary depending on the formulation method of the pharmaceutical composition or food composition, the administration method, the administration time, and/or the administration route, and may vary depending on various factors including the type and degree of the response to be achieved by the administration of the pharmaceutical composition or food composition, the type, age, weight, general health condition, symptoms or degree of the disease, sex, diet, excretion, drugs used simultaneously or at the same time in the subject, other components of the composition, and similar factors well known in the medical field, and a person having ordinary skill in the art can easily determine and prescribe an effective dosage for the intended treatment.

The pharmaceutical composition or food composition according to the present application may be administered once a day or may be administered in several divided doses. The composition may be administered as an individual therapeutic agent or in combination with other therapeutic agents and may be administered sequentially or simultaneously with conventional therapeutic agents. Taking all of the above factors into consideration, it may be administered in an amount that can obtain the maximum effect with the minimum amount without causing side effects.

For example, the composition according to the present application may be administered in a daily dosage of, but not limited to, 0.001 to 10,000 mg, 0.001 to 5,000 mg, 0.001 to 1,000 mg, 0.001 to 500 mg, 0.001 to 300 mg, 0.001 to 100 mg, 0.001 to 50 mg, 0.001 to 30 mg, 0.001 to 10 mg, 0.001 to 5 mg, 0.001 to 1 mg, 0.001 to 0.5 mg, 0.001 to 0.1 mg, 0.001 to 0.05 mg, 0.001 to 0.01 mg, 0.01 to 10,000 mg, 0.01 to 5,000 mg, 0.01 to 1,000 mg, 0.01 to 500 mg, 0.01 to 300 mg, 0.01 to 100 mg, 0.01 to 50 mg, 0.01 to 30 mg, 0.01 to 10 mg, 0.01 to 5 mg, 0.01 to 1 mg, 0.01 to 0.5 mg, 0.01 to 0.1 mg, 0.01 to 0.05 mg, 0.1 to 10,000 mg, 0.1 to 5,000 mg, 0.1 to 1,000 mg, 0.1 to 500 mg, 0.1 to 300 mg, 0.1 to 200 mg, 0.1 to 100 mg, 0.1 to 50 mg, 0.1 to 30 mg, 0.1 to 10 mg, 0.1 to 5 mg, 0.1 to 1 mg, 0.1 to 0.5 mg, 1 to 10,000 mg, 1 to 5,000 mg, 1 to 1,000 mg, 1 to 500 mg, 1 to 300 mg, 1 to 200 mg, 1 to 100 mg, 1 to 50 mg, 1 to 10 mg, 1 to 5 mg, 10 to 10,000 mg, 10 to 5,000 mg, 10 to 1,000 mg, 10 to 500 mg, 10 to 300 mg, 10 to 200 mg, 10 to 100 mg, 10 to 50 mg, 10 to 40 mg, 10 to 30 mg, 10 to 20 mg, 100 to 10,000 mg, 100 to 5,000 mg, 100 to 1,000 mg, 100 to 500 mg, 100 to 300 mg, or 100 to 200 mg per 1 kg of body weight. For example, the daily dosage of the composition according to the present application may be 0.001 to 10 g/day, 0.001 to 5 g/day, 0.01 to 10 g/day, or 0.01 to 5 g/day based on oral administration to an adult patient. In addition, the total daily dosage may be divided and administered continuously or discontinuously as needed.

Another embodiment of the present application relates to a method for enhancing immunity, comprising a step of administering to a subject at least one selected from the group consisting of Limosilactobacillus reuteri KBL346 strain deposited under Accession No. KCTC 15268BP, a culture of the strain, a lysate of the strain, and an extract of the strain.

Another embodiment of the present application relates to a method for preventing or treating influenza virus infection, comprising a step of administering to a subject at least one selected from the group consisting of Limosilactobacillus reuteri KBL346 strain deposited under Accession No. KCTC 15268BP, a culture of the strain, a lysate of the strain, and an extract of the strain.

### Effect of the Invention

The present application has found that oral administration of live or heat-killed Limosilactobacillus reuteri KBL346 exerts beneficial effects such as prevention of body weight loss and increase in survival rate of animals infected with influenza virus and also verified that it induced changes in major indicators related to inflammation and recovery. Furthermore, it has been verified that the strain according to an embodiment of the present application exhibits immune-enhancing activity. Accordingly, the strain possesses robust immune-enhancing activity and anti-influenza efficacy and can be widely applied in the development of health functional foods or therapeutics for the prevention and improvement of influenza-related infectious diseases or immune-related disorders.

### BRIEF DESCRIPTION OF THE DRAWINGS

FIG. 1 is a drawing showing the cytotoxicity of the strain according to an embodiment of the present application.

FIGS. 2A and 2B are drawings showing that the phagocytic activity of macrophages was enhanced by the strain according to an embodiment of the present application.

FIG. 3A is a drawing showing that the expression of inducible nitric oxide synthase (iNOS) in macrophages was increased by the strain according to an embodiment of the present application.

FIG. 3B is a drawing showing that the NO secretion in macrophages was increased by the strain according to an embodiment of the present application.

FIG. 4A is a drawing showing that the expression of COX-2 in macrophages was increased by the strain according to an embodiment of the present application.

FIG. 4B is a drawing showing that the secretion of PGE2 in macrophages was increased by the strain according to an embodiment of the present application.

FIGS. 5A and 5B are drawings showing that cytokine production in macrophages was increased by the strain according to an embodiment of the present application.

FIG. 6 is a drawing showing that the expression of NF-κB, and the expression and phosphorylation levels of IκBα in macrophages were increased by the strain according to an embodiment of the present application.

FIG. 7 is a drawing showing that the phosphorylation of c-Jun N-terminal kinases (JNK), extracellular signal-regulated kinase (ERK), and p38 proteins in macrophages were increased by the strain according to an embodiment of the present application.

FIG. 8A is a drawing showing the change in body weight in an influenza-infected animal model which was administered live bacteria of the strain at 0.5 of LD₅₀.

FIG. 8B is a drawing showing the body weight on day 7 post-infection, compared to body weight on the day of infection as a reference, in an influenza-infected animal model which was administered live bacteria of the strain at 0.5 of LD₅₀.

FIG. 8C is a drawing showing the survival rate in an influenza-infected animal model which was administered live bacteria of the strain at 0.5 of LD₅₀.

FIG. 9A is a drawing showing the change in body weight in an influenza-infected animal model which was administered heat-inactivated bacteria of the strain at 0.5 of LD₅₀.

FIG. 9B is a drawing showing the body weight on day 7 post-infection, compared to body weight on the day of infection as a reference, in an influenza-infected animal model which was administered with heat-inactivated bacteria of the strain at 0.5 of LD₅₀.

FIG. 9C is a drawing showing the survival rate in an influenza-infected animal model which was administered with heat-inactivated bacteria of the strain at 0.5 of LD₅₀.

FIG. 10A is a drawing showing the change in body weight in an influenza-infected animal model which was administered with the strain according to an embodiment of the present application at 4 of LD₅₀.

FIG. 10B is a drawing showing the body weight on day 7 post-infection, compared to body weight on the day of infection as a reference, in an influenza-infected animal model which was administered with the strain according to an embodiment of the present application at 4 of LD₅₀.

FIG. 10C is a drawing showing the survival rate in an influenza-infected animal model which was administered with the strain according to an embodiment of the present application at 4 of LD₅₀.

FIG. 11A is a drawing showing the expression of IFN-γ in lung tissue of an influenza-infected animal model administered with the strain according to an embodiment of the present application.

FIG. 11B is a drawing showing the expression of toll-like receptor 2 (TLR2) in lung tissue of an influenza-infected animal model administered with the strain according to an embodiment of the present application.

FIG. 11C is a drawing showing the expression of a disintegrin and metalloproteinase with thrombospondin motifs 4 (ADAMTS4) in lung tissue of an influenza-infected animal model administered with the strain according to an embodiment of the present application.

FIG. 12 is a drawing showing the blood IgG concentration in an influenza-infected animal model administered with the strain according to an embodiment of the present application.

FIG. 13 is a drawing showing the histological observation of lung tissue in an influenza-infected animal model administered with the strain according to an embodiment of the present application.

FIG. 14A is a drawing showing the prevention of body weight loss in an immunosuppressed animal model administered with the strain according to an embodiment of the present application.

FIG. 14B is a drawing showing the increase in monocyte levels in blood in an immunosuppressed animal model administered with the strain according to an embodiment of the present application.

### MODE FOR INVENTION

Hereinafter, the present application will be described in more detail with reference to the following Examples. However, these Examples are only intended to illustrate the present application, and the scope of the present application is not limited by these Examples.

### Example 1. Evaluation of Cytotoxicity of Limosilactobacillus reuteri KBL346 Strain

A fecal sample from a 3-month-old infant was provided through Seoul Samsung Hospital. The sample was inoculated onto a selective medium (TOS-propionate agar medium; 43314 Transgalactosylated oligosaccharide agar medium, Fluka), cultured for 48 hours in an anaerobic environment at 37° C., and pure cultured using the colony-picking method.

The isolated strain was cultured in an anaerobic environment in a 37° C. CO₂ incubator for one day and centrifuged at 4° C. and 13,000 rpm to obtain the bacterial pellet only. Then, 0.2 μL of the bacterial pellet was added to 25 μL of lysis buffer (containing pH 10; 2.5 M NaCl, 100 mM EDTA, 10 mM Trizma base, and 1% Triton X-100) and heat-treated at 95° C. for 10 minutes to obtain the bacterial genome. Then, the V4 region of the 16S rRNA gene was amplified by polymerase chain reaction using a G-Taq PCR kit to obtain a PCR product. The nucleic acid sequences of the primers used are shown in Table 1. Among the nucleic acid sequences of the primers below, “M” is selected from A or C, and “Y” is selected from C or T.

TABLE 1

| | | SEQ ID |
|---|---|---|
| Classification | Sequence(5′→3′) | NO |
| 27F forward primer | AGAGTTTGATCMTGGCTCAG | 1 |
| 1492R reverse primer | TACGGYTACCTTGTTACGACTT | 2 |
| Limosilactobacillus | AGTTTGGAACATGGCTCAGGATGAACGCCGGCG | 3 |
| reuteri 16S rRNA | GTGTGCCTAATACATGCAAGTCGTACGCACTGG | |
| | CCCAACTGATTGATGGTGCTTGCACCTGATTGA | |
| | CGATGGATCACCAGTGAGTGGCGGACGGGTGA | |
| | GTAACACGTAGGTAACCTGCCCCGGAGCGGGG | |
| | GATAACATTTGGAAACAGATGCTAATACCGCATA | |
| | ACAACAAAAGCCACATGGCTTTTGTTTGAAAGAT | |
| | GGCTTTGGCTATCACTCTGGGATGGACCTGCGG | |
| | TGCATTAGCTAGTTGGTAAGGTAACGGCTTACC | |
| | AAGGCGATGATGCATAGCCGAGTTGAGAGACTG | |
| | ATCGGCCACAATGGAACTGAGACACGGTCCATA | |
| | CTCCTACGGGAGGCAGCAGTAGGGAATCTTCCA | |
| | CAATGGGCGCAAGCCTGATGGAGCAACACCGC | |
| | GTGAGTGAAGAAGGGTTTCGGCTCGTAAAGCTC | |
| | TGTTGTTGGAGAAGAACGTGCGTGAGAGTAACT | |
| | GTTCACGCAGTGACGGTATCCAACCAGAAAGTC | |
| | ACGGCTAACTACGTGCCAGCAGCCGCGGTAATA | |
| | CGTAGGTGGCAAGCGTTATCCGGATTTATTGGG | |
| | CGTAAAGCGAGCGCAGGCGGTTGCTTAGGTCT | |
| | GATGTGAAAGCCTTCGGCTTAACCGAAGAAGTG | |
| | CATCGGAAACCGGGCGACTTGAGTGCAGAAGA | |
| | GGACAGTGGAACTCCATGTGTAGCGGTGGAATG | |
| | CGTAGATATATGGAAGAACACCAGTGGCGAAGG | |
| | CGGCTGTCTGGTCTGCAACTGACGCTGAGGCTC | |
| | GAAAGCATGGGTAGCGAACAGGATTAGATACCC | |
| | TGGTAGTCCATGCCGTAAACGATGAGTGCTAGG | |
| | TGTTGGAGGGTTTCCGCCCTTCAGTGCCGGAGC | |
| | TAACGCATTAAGCACTCCGCCTGGGGAGTACGA | |
| | CCGCAAGGTTGAAACTCAAAGGAATTGACGGGG | |
| | GCCCGCACAAGCGGTGGAGCATGTGGTTTAATT | |
| | CGAAGCTACGCGAAGAACCTTACCAGGTCTTGA | |
| | CATCTTGCGCTAACCTTAGAGATAAGGCGTTCC | |
| | CTTCGGGGACGCAATGACAGGTGGTGCATGGT | |
| | CGTCGTCAGCTCGTGTCGTGAGATGTTGGGTTA | |
| | AGTCCCGCAACGAGCGCAACCCTTGTTACTAGT | |
| | TGCCAGCATTAAGTTGGGCACTCTAGTGAGACT | |
| | GCCGGTGACAAACCGGAGGAAGGTGGGGACGA | |
| | CGTCAGATCATCATGCCCCTTATGACCTGGGCT | |
| | ACACACGTGCTACAATGGACGGTACAACGAGTC | |
| | GCAAGCTCGCGAGAGTAAGCTAATCTCTTAAAG | |
| | CCGTTCTCAGTTCGGACTGTAGGCTGCAACTCG | |
| | CCTACACGAAGTCGGAATCGCTAGTAATCGCGG | |
| | ATCAGCATGCCGCGGTGAATACGTTCCCGGGCC | |
| | TTGTACACACCGCCCGTCACACCATGGGAGTTT | |
| | GTAACGCCCAAAGTCGGTGGCCTAACCTTTATG | |
| | GAGGGAGCCGCCTAAGGCGGGACAGATGACTG | |
| | GGGTGAAGTCGTACAGGGGGAAACCCGT | |
| Lacticaseibacillus | TCTGATTATTGAAAGGTGCTTGCTTCTTGATTTA | 4 |
| rhamnosus 16S rRNA | ATTTTGAACGAGTGGCGGACGGGTGAGTAACAC | |
| | GTGGGTAACCTGCCCTTAAGTGGGGGATAACAT | |
| | TTGGAAACAGATGCTAATACCGCATAAATCCAAG | |
| | AACCGCATGGTTCTTGGCTGAAAGATGGCGTAA | |
| | GCTATCGCTTTTGGATGGACCCGCGGCGTATTA | |
| | GCTAGTTGGTGAGGTAACGGCTCACCAAGGCAA | |
| | TGATACGTAGCCGAACTGAGAGGTTGATCGGCC | |
| | ACATTGGGACTGAGACACGGCCCAAACTCCTAC | |
| | GGGAGGCAGCAGTAGGGAATCTTCCACAATGGA | |
| | CGCAAGTCTGATGGAGCAACGCCGCGTGAGTG | |
| | AAGAAGGCTTTCGGGTCGTAAAACTCTGTTGTT | |
| | GGAGAAGAATGGTCGGCAGAGTAACTGTTGTCG | |
| | GCGTGACGGTATCCAACCAGAAAGCCACGGCTA | |
| | ACTACGTGCCAGCAGCCGCGGTAATACGTAGGT | |
| | GGCAAGCGTTATCCGGATTTATTGGGCGTAAAG | |
| | CGAGCGCAGGCGGTTTTTTAAGTCTGATGTGAA | |
| | AGCCCTCGGCTTAACCGAGGAAGTGCATCGGAA | |
| | ACTGGGAAACTTGAGTGCAGAAGAGGACAGTGG | |
| | AACTCCATGTGTAGCGGTGAAATGCGTAGATAT | |
| | ATGGAAGAACACCAGTGGCGAAGGCGGCTGTC | |
| | TGGTCTGTAACTGACGCTGAGGCTCGAAAGCAT | |
| | GGGTAGCGAACAGGATTAGATACCCTGGTAGTC | |
| | CATGCCGTAAACGATGAATGCTAGGTGTTGGAG | |
| | GGTTTCCGCCCTTCAGTGCCGCAGCTAACGCAT | |
| | TAAGCATTCCGCCTGGGGAGTACGACCGCAAG | |
| | GTTGAAACTCAAAGGAATTGACGGGGGCCCGCA | |
| | CAAGCGGTGGAGCATGTGGTTTAATTCGAAGCA | |
| | ACGCGAAGAACCTTACCAGGTCTTGACATCTTTT | |
| | GATCACCTGAGAGATCAGGTTTCCCCTTCGGGG | |
| | GCAAAATGACAGGTGGTGCATGGTTGTCGTCAG | |
| | CTCGTGTCGTGAGATGTTGGGTTAAGTCCCGCA | |
| | ACGAGCGCAACCCTTATGACTAGTTGCCAGCAT | |
| | TTAGTTGGGCACTCTAGTAAGACTGCCGGTGAC | |
| | AAACCGGAGGAAGGTGGGGATGACGTCAAATCA | |
| | TCATGCCCCTTATGACCTGGGCTACACACGTGC | |
| | TACAATGGATGGTACAACGAGTTGCGAGACCGC | |
| | GAGGTCAAGCTAATCTCTTAAAGCCATTCTCAGT | |
| | TCGGACTGTAGGCTGCAACTCGCCTACACGAAG | |
| | TCGGAATCGCTAGTAATCGCGGATCAGCACGCC | |
| | GCGGTGAATACGTTCCCGGGCCTTGTACACACC | |
| | GCCCGTCACACCATGAGAGTTTGTAACACCCGA | |
| | AGCCGGTGGCGTAACCCTTTTAGGGAGCGAGC | |

CGTCTAAGGTGGGACAAATGATTAGGGTGAAGT
CGTAAC

The PCR product was purified using an Ultra Clean PCR clean-up Kit (Mobio Laboratories Inc.) and the genetic sequence analysis was requested to Macrogen to identify the strains. As a result, they were identified as Limosilactobacillus reuteri strains and Lacticaseibacillus rhamnosus strains and named for Limosilactobacillus reuteri KBL346 and Lacticaseibacillus rhamnosus KBL352, respectively. The 16S rRNA sequence of the Limosilactobacillus reuteri KBL346 strain is shown in SEQ ID NO: 3, and the 16S rRNA sequence of the Lacticaseibacillus rhamnosus KBL352 strain is shown in SEQ ID NO: 4.

The two strains were deposited with the Korean Collection for Type Cultures of the Korea Research Institute of Bioscience and Biotechnology, an international depository institution under the Budapest Treaty, and were assigned accession numbers KCTC 15268BP and KCTC 15267BP, respectively.

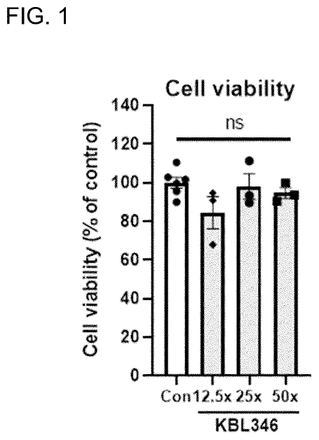

The results of evaluating the cell viability by treating the mouse macrophage cell line RAW264.7 with live Limosilactobacillus reuteri KBL346 at a concentration of 12.5 to 50 times are shown in FIG. 1. As shown in FIG. 1, even when treated with concentration up to 50 times compared to that of RAW264.7 cells, the viability of macrophages was not affected, it was confirmed that the Limosilactobacillus reuteri KBL346 strain could be used without cytotoxicity.

### Example 2. Enhancement Effect of Phagocytic Activity of Macrophages

By using Candida albicans MYA-4788 (hereinafter referred to as C. albicans), enhancing effect of the strain according to an embodiment of the present application on macrophage phagocytosis was analyzed. Specifically, the macrophage cell line RAW264.7 was treated with live Limosilactobacillus reuteri KBL346 at a ratio of 50 times, and co-cultured for 24 hours. Then, the RAW264.7 cells were washed twice with 1×PBS. As a positive control, lipopolysaccharide (LPS) was treated to RAW264.7 cells at a concentration of 10 ng/ml.

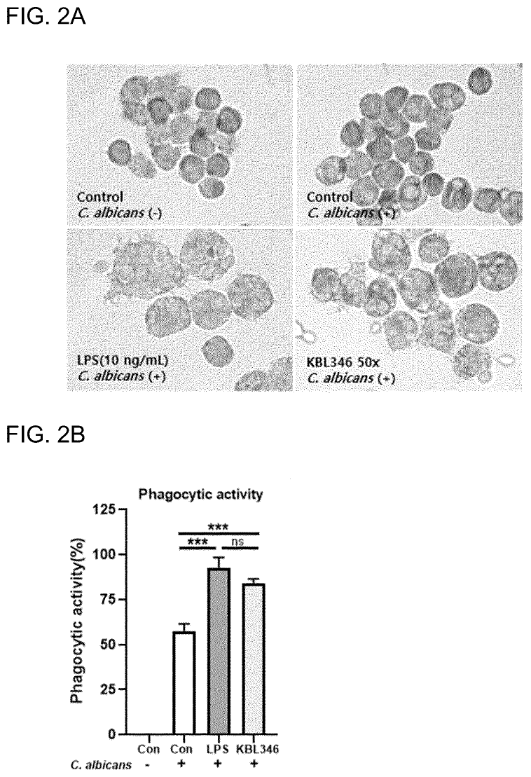

Prepared RAW264.7 cells were treated with C. albicans in yeast state at a 10-fold level and cultured at 37° C. for 1 hour. After completing co-culture with C. albicans, RAW264.7 cells were washed twice with 1×PBS, fixed by treating with 2.5% glutaraldehyde and stained by treating with 0.5% methylene blue dye. The stained cells were observed under a microscope, and if one or more C. albicans were observed inside the macrophage, it was considered that phagocytosis had occurred. The results of the microscopic observation are shown in FIG. 2A, and the phagocytic activity of the macrophages is calculated as follows and shown in FIG. 2B and Table 2:

$$Phagocytic Activity ( \% ) = ( Number of cells with induced phagocytosis ) \text{} / ( Number of observed cells ) \times 100$$

TABLE 2

| Classification | Phagocytic Activity (%) |
|---|---|
| Untreated group | 57.4 |
| KBL346 | 84.2 |

As shown in FIGS. 2A and 2B and Table 2, a significant increase in the phagocytic activity of macrophages was observed upon treatment with live Limosilactobacillus reuteri KBL346.

### Example 3. Increasing Effect of iNOS Expression and NO Secretion in Macrophages

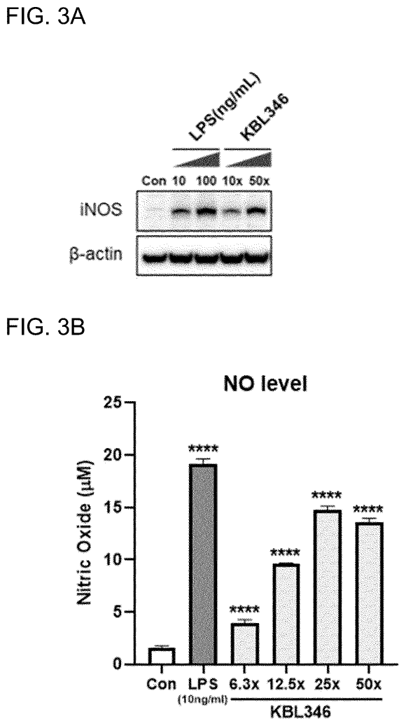

After treating the mouse macrophage cell line RAW264.7 with live bacteria of Limosilactobacillus reuteri KBL346 at a concentration of 6.3 to 50 times, the expression level of iNOS, a protein involved in the production of NO, and the amount of NO secreted by macrophages were measured. As a positive control, LPS was treated at a concentration of 10 ng/ml or 100 ng/ml, and the expression level of β-actin as a housekeeping protein was observed together to show that the protein used was constant in all samples. The expression of iNOS is shown in FIG. 3A and the amount of NO secreted by macrophages is shown in FIG. 3B and Table 3.

As shown in FIG. 3A, FIG. 3B, and Table 3, the iNOS expression and the amount of NO secreted increased depending on the treatment level of strain according to an embodiment of the present application, which referred that the strain according to an embodiment of the present application was an immune stimulant activating immune cells.

TABLE 3

| Classification | NO concentration (μM) |
|---|---|
| Untreated group | 1.6 |
| LPS 10 ng/ml | 19.2 |
| KBL346 6.3x | 4.0 |
| KBL346 12.5x | 9.6 |
| KBL346 25x | 14.8 |
| KBL346 50x | 13.6 |

### Example 4. Increasing effect of COX-2 expression and PGE2 secretion in macrophages

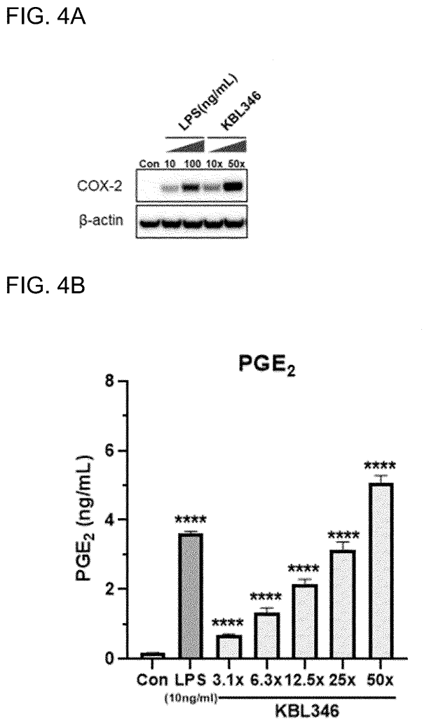

The expression of COX-2, as a protein involved in PGE2 production, and the amount of PGE2 secreted by macrophages were measured after treating the mouse macrophage cell line RAW264.7 with live bacteria of Limosilactobacillus reuteri KBL346 at a concentration gradient of 3.1 to 50 times. As a positive control, LPS was treated at a concentration of 10 ng/ml or 100 ng/ml. The expression level of β-actin, as a housekeeping protein, was observed together to show that the protein used was constant in all samples. The expression of COX-2 is shown in FIG. 4A, and the amount of PGE2 secreted by macrophages is shown in FIG. 4B.

As shown in FIG. 4A, FIG. 4B, and Table 4, the expression level of COX-2 and the amount of PGE2 secreted increased depending on the treatment level of strain according to an embodiment of the present application, which referred that the strain according to an embodiment of the present application was an immune stimulant that activates immune cells.

TABLE 4

| Classification | PGE₂ concentration (ng/ml) |
|---|---|
| Untreated group | 0.1655 |
| LPS 10 ng/ml | 3.5921 |
| KBL346 3.1x | 0.6719 |
| KBL346 6.3x | 1.3416 |
| KBL346 12.5x | 2.1437 |
| KBL346 25x | 3.1399 |
| KBL346 50x | 5.0661 |

### Example 5. Increasing effect of cytokine production in macrophages

The mouse macrophage cell line RAW264.7 was treated with live bacteria of Limosilactobacillus reuteri KBL346 at a concentration of 12.5 to 50 times. The production of TNF-α and IL-6 in macrophages were measured. LPS was treated at a concentration of 10 ng/mL as a positive control.

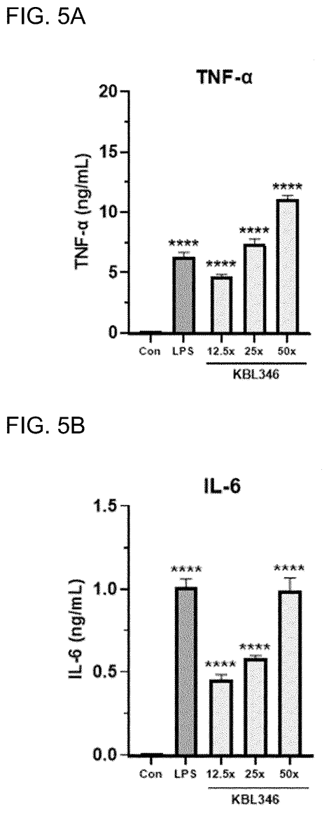

As shown in FIG. 5A, FIG. 5B, and Table 5, of TNF-α and IL-6 in the macrophages increased depending on the treatment level of strain according to an embodiment of the present application, which referred that the strain according to an embodiment of the present application had an enhancement effect of immune function.

TABLE 5

| Classification | TNF-α (ng/ml) | IL-6 (ng/ml) |
|---|---|---|
| LPS | 6.3 | 1.01 |
| Untreated group | 0 | 0 |
| KBL346 12.5x | 4.7 | 0.45 |
| KBL346 25x | 7.3 | 0.58 |
| KBL346 50x | 11.1 | 0.99 |

### Example 6. Increasing effect of expression and phosphorylation of NF-κB and IκBα in macrophages

Mouse macrophage cell line RAW264.7 was treated with live bacteria of Limosilactobacillus reuteri KBL346 at a concentration of 10-fold or 50-fold. The expression and phosphorylation of NF-κB and IκBα proteins were tested. As a control, LPS was treated at 10 ng/ml or 100 ng/ml, and the expression level of β-actin, a housekeeping protein, was observed together to show that the protein used was constant in all samples.

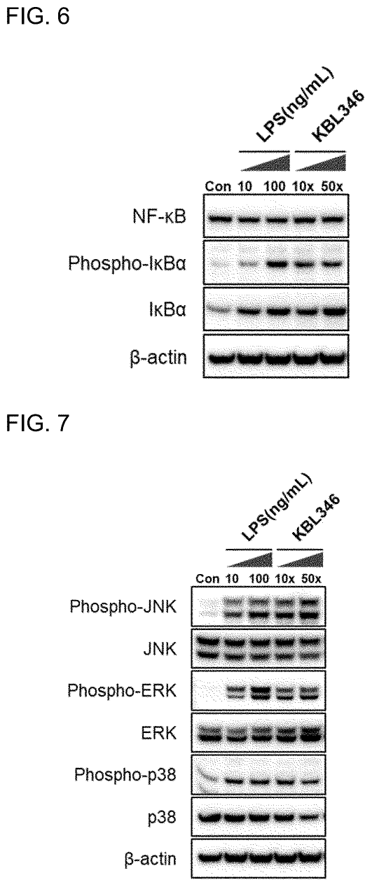

As shown in FIG. 6, the expression of NF-κB and the expression and phosphorylation of IκBα were increased by treatment of the strain according to an embodiment of the present application, and it was confirmed that the immune ability was activated.

### Example 7. Effect on activation of MAPKs in macrophages

Mouse macrophage cell line RAW264.7 was treated with Limosilactobacillus reuteri KBL346 and the expression and phosphorylation levels of MAPKs (p38, ERK, JNK, etc.) signaling pathways, which are important signaling pathways for immune cell activation, were evaluated by promoting NO production in immune cells and producing cytokines such as TNF-α and IL-6. As a control, LPS was treated at 10 ng/ml or 100 ng/ml, and the expression level of β-actin, a housekeeping protein, was observed together to show that the protein used was constant in all samples.

As shown in FIG. 7, when treating with the strain according to an embodiment of the present application, the phosphorylation of JNK, ERK, and p38 proteins increased, and it was confirmed that the immune ability was activated.

### Example 8. Improvement Effect on of Disease Severity and Survival Rate in an Influenza-Infected Animal Model at 0.5 LD₅₀ (Lethal Dose 50%) (1)

The freeze-dried powder of the Limosilactobacillus reuteri KBL346 strain and the Lacticaseibacillus rhamnosus strain deposited under accession number KCTC 15267BP obtained in Example 1 was prepared, and the number of cells was verified through the CFU assay. The freeze-dried powder of the culture solution was diluted in PBS so as to obtain 5.0×10⁹ CFU/ml of concentration for each strain.

Eight-week-old female Balb/c mice were orally administered once a day until the end of the experiment with 200 μL of the diluted solution so that each strain was 1.0×10⁹ CFU. After administering each strain for one week, infection was induced by injecting a PR8 influenza virus at a 0.5 lethal dose (0.5 LD₅₀) through the nasal cavity after respiratory anesthesia. Even after infection, each strain was orally administered once a day until the end of the experiment, with the strains at concentration of 1.0×10⁹ CFU for each strain.

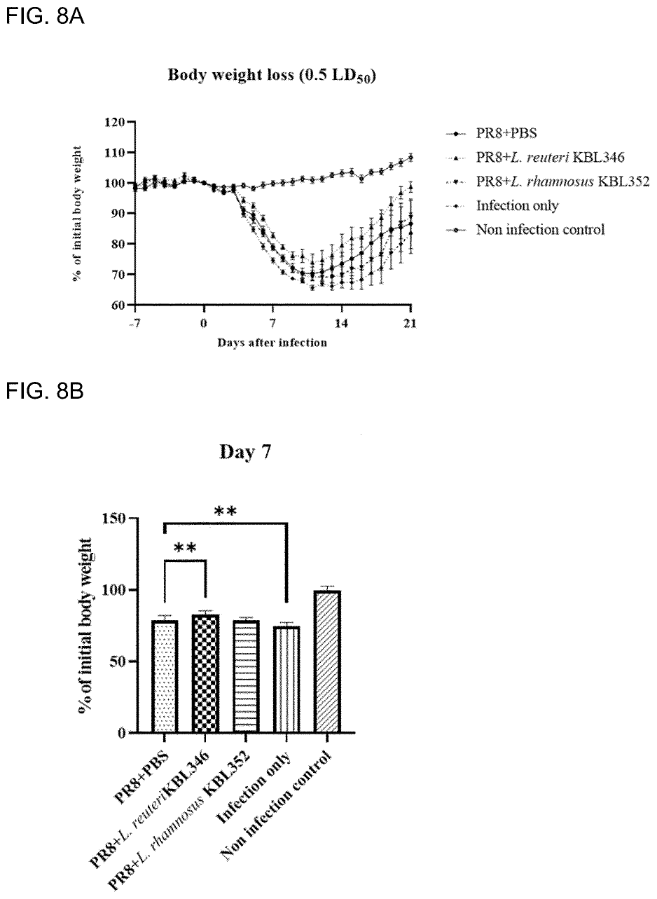

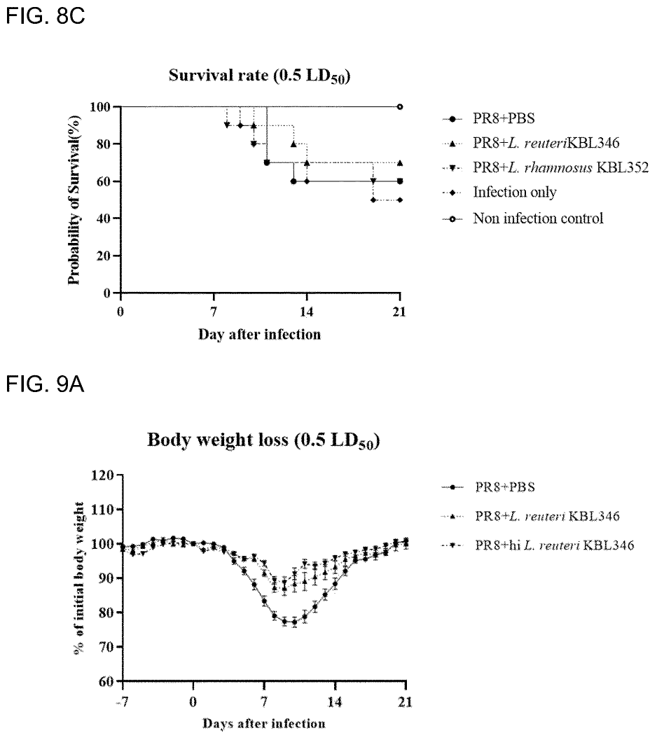

Since the start of Limosilactobacillus reuteri KBL346 strain administration, the body weight of the mice was measured every day and the change in body weight based on 100% of the body weight on the day of influenza infection is shown in FIG. 8A. The body weight on the 7th day of infection compared to the day of influenza infection is shown in FIG. 8B and Table 6. In addition, the survival rate of mice after the end of the experiment is shown in FIG. 8C and Table 7.

TABLE 6

| Classification | Change of body weight (%) |
|---|---|
| PR8 + PBS | 78.9 |
| KBL346 | 82.8 |
| KBL352 | 78.7 |
| Infection only | 74.6 |
| Non-infection | 99.8 |

TABLE 7

| Classification | Survival rate (%) |
|---|---|
| PR8 + PBS | 60 |
| KBL346 | 70 |
| KBL352 | 60 |
| Infection only | 50 |
| Non-infection | 100 |

As shown in FIG. 8A, FIG. 8B and Table 6, the Limosilactobacillus reuteri KBL346 strain according to an embodiment of the present application prevented weight loss caused by influenza virus infection and was excellent in reducing the severity of influenza virus infection. In addition, as shown in FIG. 8C and Table 7, the Limosilactobacillus reuteri KBL346 strain according to an embodiment of the present application significantly reduced the mortality rate of influenza virus infection.

### Example 9. Improvement Effect of Disease Severity and Survival Rate in an Influenza-Infected Animal Model at 0.5 LD₅₀ (2)

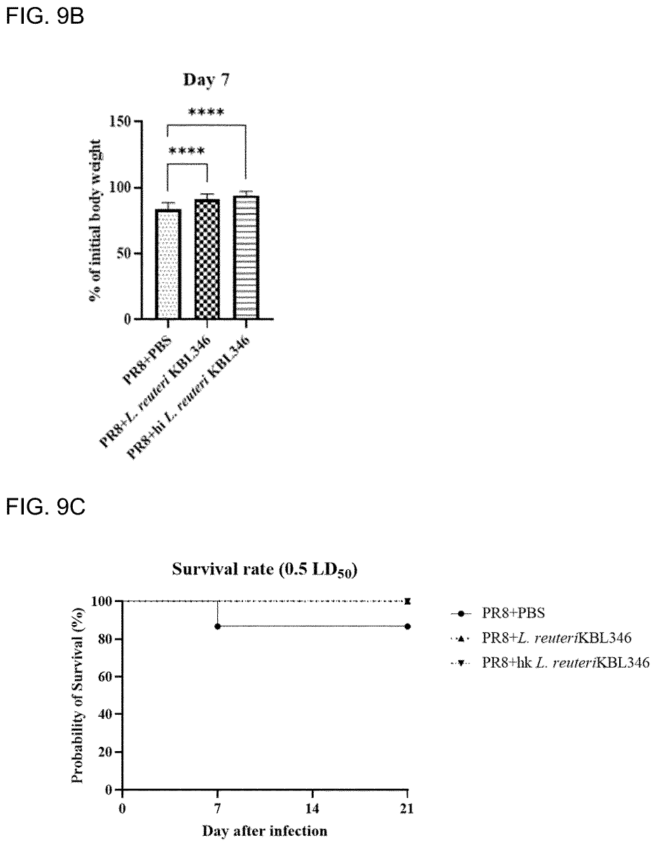

Limosilactobacillus reuteri KBL346 strain obtained in Example 1 was inactivated by heat treatment at 55° C. for 30 minutes, and then the experiment was performed with heat-inactivated or live Limosilactobacillus reuteri KBL346 in the same manner as in Example 8. The change in body weight based on 100% of the body weight on the day of influenza infection is shown in FIG. 9A, the result of body weight on the 7th day of infection compared to the day of influenza infection are shown in FIG. 9B and Table 8, and the survival rate of the mice after the end of the experiment is shown in FIG. 9C and Table 9.

TABLE 8

| Classification | Change of body weight (%) |
|---|---|
| PBS | 83.4 |
| KBL346 | 91.5 |
| Heat-inactivated | 94.3 |
| KBL346 | |

TABLE 9

| Classification | Survival rate (%) |
|---|---|
| PBS | 86.7 |
| KBL346 | 100.0 |
| Heat-inactivated | 100.0 |
| KBL346 | |

As shown in FIGS. 9A and 9B, and Table 8, the Limosilactobacillus reuteri KBL346 strain according to an embodiment of the present application prevented weight loss caused by influenza virus infection and lowered the severity of influenza virus infection even when using heat-inactivated strain, and in particular, the heat-inactivated strain maintained an effect equivalent to that of a live strain. Therefore, for use in enhancing immunity or for anti-influenza purposes, there is no need to maintain the strain according to an embodiment of the present application in a live state, thereby reducing the cost for maintaining the live state. In addition, as shown in FIG. 9C and Table 9, the heat-inactivated Limosilactobacillus reuteri KBL346 strain according to an embodiment of the present application significantly lowered the mortality rate of influenza virus infection.

### Example 10. Improvement Effect of Disease Severity and Survival Rate in a Mouse Model of Influenza Infection Induced at 4 LD₅₀

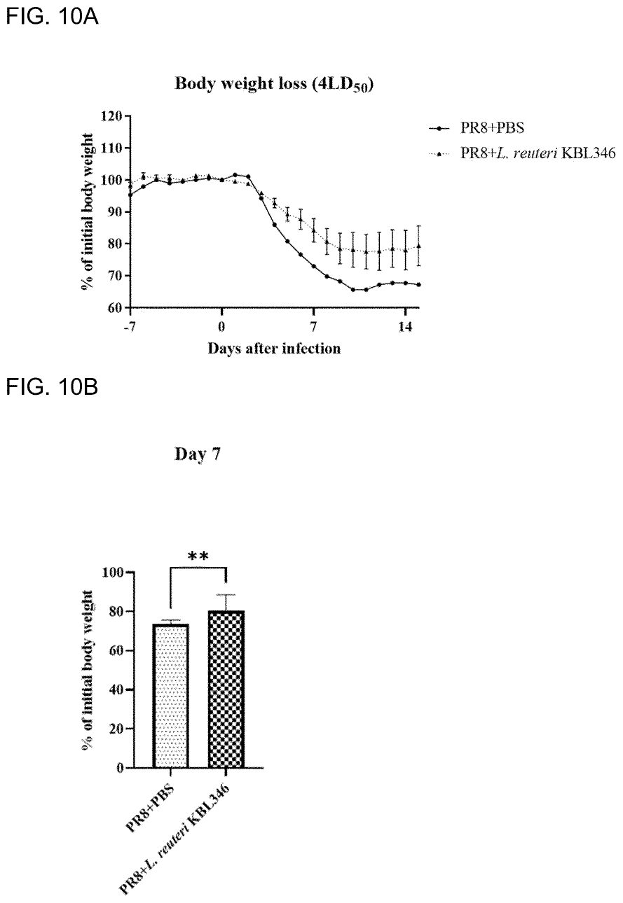

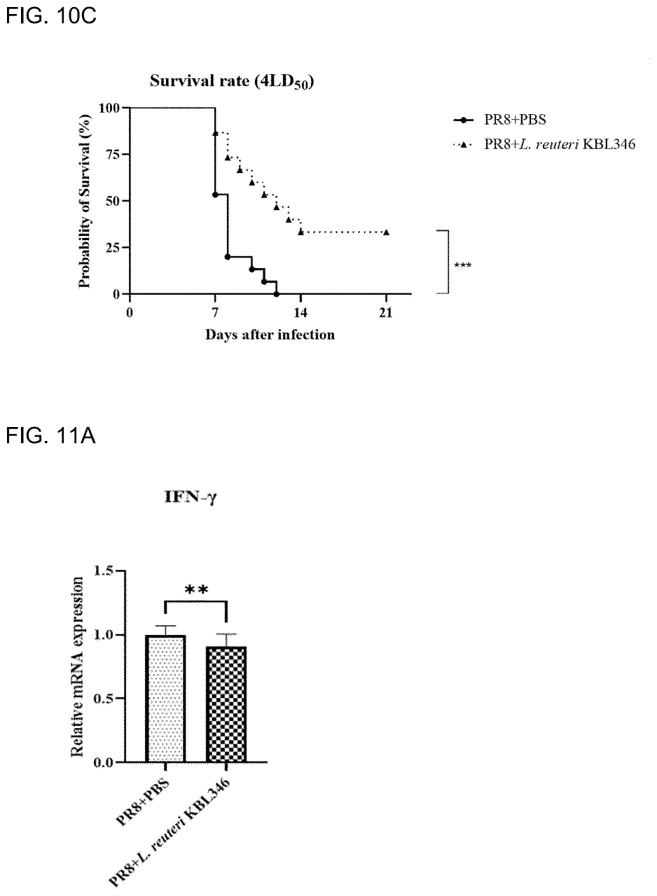

The experiment was conducted in the same manner as in Example 8, but infection was induced by administering 4 LD₅₀ PR8 influenza virus after administering the Limosilactobacillus reuteri KBL346 strain for one week. The change in body weight based on 100% of the body weight on the day of influenza infection is shown in FIG. 10C, the result of comparing the body weight on the 7th day of infection to the day of influenza infection is shown in FIG. 10C and Table 10, and the survival rate of the mice after the end of the experiment is shown in FIG. 10C and Table 11.

TABLE 10

| Classification | Change of body weight (%) |
|---|---|
| PBS | 73.6 |
| KBL346 | 80.3 |

TABLE 11

| Classification | Survival rate (%) |
|---|---|
| PBS | 0 |
| KBL346 | 33.3 |

As shown in FIGS. 10A to 10C and Tables 10 to 11, the Limosilactobacillus reuteri KBL346 strain according to an embodiment of the present application showed the effect of significantly reducing the severity and mortality rate even when infected with 4 LD₅₀ of an influenza virus.

### Example 11. Alleviation Effect of Pulmonary Inflammation

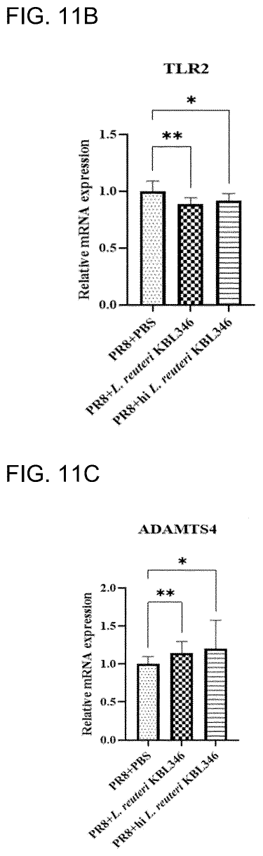

From an animal model infected with 0.5 LD₅₀ of influenza virus in Examples 8 and 9, lung tissues were collected on day 7 after infection. Gene expressions of the inflammatory cytokine IFN-γ, the receptor TLR2 that can mediate inflammatory responses, and ADAMTS4 that induces recovery in the infection situation were confirmed through qPCR. Changes of these gene expressions are shown in FIGS. 11A to 11C and Table 12 based on the control group (PBS).

TABLE 12

| Classification | IFN-γ | TLR2 | ADAMTS4 |
|---|---|---|---|
| PBS | 1.0 | 1.0 | 1.0 |
| KBL346 | 0.907 | 0.886 | 1.142 |
| Heat-inactivated | N/A | 0.915 | 1.203 |
| KBL346 | | | |

As shown in FIGS. 11A to 11C and Table 12, it was confirmed that IFN-γ and TLR2 was suppressed and ADAMTS4 was induced in the groups administered with the live and heat-treated Limosilactobacillus reuteri KBL346 strain according to an embodiment of the present application.

### Example 12. Reducing Effect of Blood Antibody (IgG) Concentration

From the animal models infected with 0.5 LD₅₀ of influenza virus in Example 9, blood of the animal model administered with the heat-inactivated strain was collected on the 7th day of infection, and the IgG concentration was tested through ELISA. IgG specifically binds to the surface protein haemagglutinin (HA) of the PR8 influenza virus, and the blood IgG concentration indicates the amount of influenza virus in the plasma.

As shown in FIG. 12 and Table 13, it was confirmed that the blood IgG concentration decreased when the Limosilactobacillus reuteri KBL346 strain according to an embodiment of the present application was administered to the influenza-infected model. Therefore, the Limosilactobacillus reuteri KBL346 strain according to an embodiment of the present application has the effect of inducing rapid recovery from influenza virus infection.

TABLE 13

| Classification | IgG (Absorbance/450 nm) |
|---|---|
| PBS | 0.640 |
| Heat-inactivated | 0.390 |
| KBL346 | |

### Example 13. Histopathological Improvement Effect

From an animal model infected with 0.5 LD₅₀ of influenza virus in Examples 8 and 9, lung tissues were collected on the 7th day of infection, fixed using 10% neutral buffered formalin, subjected to (formalin fixed paraffin embedded (FPPE)), sectioned into 4 μm thickness, and stained with hematoxylin & eosin. An animal model administered with 20 mg/kg of Oseltamivir for 1 day before influenza virus infection was used as a positive control. The results of lung tissue observation are shown in FIG. 13 (A: Group administered with PBS, B: Group administered with Oseltamivir, C: Group administered with Limosilactobacillus reuteri KBL346, D: Group administered with heat-inactivated Limosilactobacillus reuteri KBL346).

As shown in FIG. 13a, the group administered with PBS showed excessive infiltration of immune cells, which appeared as blue dots due to lung infection. Histopathological symptoms such as rupture, bronchial epithelial necrosis, and atelectasis were observed. On the other hand, as shown in FIGS. 13c and 13d, in the group administered with strain according to an embodiment of the present application, such symptoms were relatively less, and lung tissue was well maintained at a level equivalent to that in the group administered with Oseltamivir (FIG. 13b), which is a conventional agent for preventing and treating influenza virus.

### Example 14. Immune-Enhancing Effect

(1) Production of an Animal Model with Immunosuppression

Water, Limosilactobacillus reuteri KBL346 strain, or red ginseng extract was orally administered to 5-week-old male Balb/c mice once a day until the end of the experiment according to Table 14. On the 15th and 16th days of the experiment, cyclophosphamide was administered intraperitoneally to all mice except the naïve group at a concentration of 150 mg/kg, thereby inducing immunosuppression.

TABLE 14

| Group | Dosage | Concentration |
|---|---|---|
| Naive | water 200 μL | — |
| Control | water 200 μL | — |
| KBL346 | 200 μL | Diluted in water to 10⁹ CFU/mL |
| Red ginseng | 200 μL | 12.7 mg/g of ginsenosides |
| | (200 mg/kg) | including Rg1, Rb1, |
| | | and Rg3 was contained |

### (2) Effect of Preventing Weight Loss

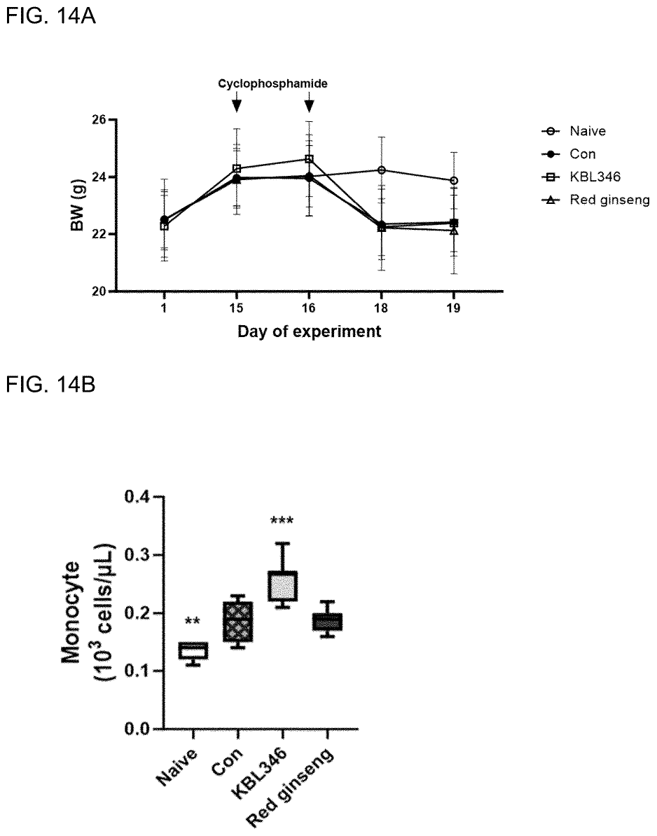

The weight of the mice before oral administration once a day from the first day of the experiment to the end was measured and shown in FIG. 14A. As shown in FIG. 14A, the Limosilactobacillus reuteri KBL346 strain according to an embodiment of the present application showed an effect of preventing weight loss caused by immunosuppression.

### (3) Effect of Increasing Monocytes

After the experiment, orbital blood was collected from each group of mice, and monocytes in the blood were measured using a blood cell analyzer, which is shown in FIG. 14B. As shown in FIG. 14B, Monocytes significantly increased in the group administered with Limosilactobacillus reuteri KBL346 strain according to an embodiment of the present application, and particularly, it showed an equivalent to or greater than that of red ginseng. Therefore, the Limosilactobacillus reuteri KBL346 strain according to an embodiment of the present application has a significantly excellent effect of increasing immunity that induces rapid recovery from immunosuppression. In FIG. 14B, * indicates p<0.05; ** indicates p<0.01; *** indicates p<0.001; and **** indicates p<0.0001, compared to the control group.

### [Accession Information]

### Depositary Authority: Korean Collection for Type Cultures (KCTC), Korea Research Institute of Bioscience and Biotechnology (KRIBB)

Accession Number: KCTC15268BP
Date of Deposit: Jan. 4, 2023

### Depositary Authority: Korean Collection for Type Cultures (KCTC), Korea Research Institute of Bioscience and Biotechnology (KRIBB)

Accession Number: KCTC15267BP
Date of Deposit: Jan. 4, 2023

1.-16. (canceled)

17. A method for enhancing immunity in a subject, the method comprising a step of administering to the subject a composition comprising a Limosilactobacillus reuteri KBL346 bacterial strain deposited under Accession No. KCTC 15268BP, a culture of the strain, a lysate of the strain, or an extract thereof.

18. The method according to claim 17, wherein the method of enhancing immunity comprises activating macrophages.

19. The method according to claim 17, wherein the composition is an immune-stimulator.

20. The method according to claim 17, wherein the strain is a live bacterium or a heat-inactivated bacterium.

21. The method according to claim 17, wherein the composition has at least one of the following characteristics:
(1) promotion of phagocytic activity of macrophages;
(2) promotion of NO secretion by macrophages;
(3) promotion of COX-2 expression in macrophages;
(4) promotion of PGE2 secretion by macrophages;
(5) promotion of cytokine production by macrophages;
(6) promotion of expression and phosphorylation of NF-κB and/or IκBα in macrophages;
(7) promotion of activation of MAPKs in macrophages;
(8) promotion of recovery from viral infection;
(9) prevention of body weight loss caused by immunosuppression; or
(10) increase in monocytes in blood.

22. A method for preventing or treating influenza virus infection in a subject, the method comprising a step of administering to the subject a composition comprising a Limosilactobacillus reuteri KBL346 bacterial strain deposited under Accession no. KCTC 15268BP, a culture of the strain, a lysate of the strain, or an extract thereof.

23. The method according to claim 22, wherein the strain is a live bacterium or a heat-inactivated bacterium.

24. The method according to claim 22, wherein the composition comprises the strain at a concentration of 1.0×10³ to 1.0×10¹⁵ CFU.

25. The method according to claim 22, wherein the composition has at least one of the following characteristics:
(1) prevention of body weight loss caused by influenza virus infection;
(2) reduction in mortality caused by influenza virus infection;
(3) alleviation of pulmonary inflammation; or
(4) reduction in serum immunoglobulin G (IgG) levels.

26. The method according to claim 22, wherein the influenza virus infection is flu, pneumonia, Reye's syndrome, acute respiratory failure, myocarditis, bronchitis, otitis media, pharyngitis, sinusitis, empyema, Guillain-Barre syndrome, or encephalitis.

27. The method according to claim 22, wherein the influenza virus infection has at least one symptom selected from the group consisting of fever, chills, nausea, myalgia, malaise, cough, dyspnea, shortness of breath, pneumonia, sputum, sore throat, headache, hemoptysis, and diarrhea.

28. A Limosilactobacillus reuteri KBL346 strain deposited under Accession No. KCTC 15268BP.

29. The strain according to claim 28, wherein the strain comprises a 16S IRNA sequence set forth in SEQ ID NO: 3.

30. A composition in the form of tablets, pills, troches, lozenges, aerosols, suspensions, powders, granules, emulsions, capsules, syrups, or elixirs, comprising the strain according to claim 28, or a culture of the strain, a lysate of the strain, or an extract thereof.

31. A food comprising the strain according to claim 28, a culture of the strain, a lysate of the strain, or an extract thereof.

32. A quasi-drug comprising the strain according to claim 28, a culture of the strain, a lysate of the strain, or an extract thereof.


SEQUENCE LISTING (4 sequences)

SEQ ID NO: 1 - DNA, 20 bp, synthetic construct
  misc_feature 1..20: note = 27F forward
  source 1..20: mol_type = other DNA
agagtttgatcmtggctcag

SEQ ID NO: 2 - DNA, 22 bp, synthetic construct
  misc_feature 1..22: note = 1492R reverse
  source 1..22: mol_type = other DNA
tacggytaccttgttacgactt

SEQ ID NO: 3 - DNA, 1540 bp, Limosilactobacillus reuteri
  misc_feature 1..1540: note = Limosilactobacillus reuteri
  source 1..1540: mol_type = genomic DNA
agtttggaacatggctcaggatgaacgccggcggtgtgcctaatacatgcaagtcgtacgcactggcccaactgattgatggtgcttgcacctgattgacgatggatcaccagtgagtggcggacgggtgagtaacacgtaggtaacctgccccggagcgggggataacatttggaaacagatgctaataccgcataacaacaaaagccacatggcttttgtttgaaagatggctttggctatcactctgggatggacctgcggtgcattagctagttggtaaggtaacggcttaccaaggcgatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaagctctgttgttggagaagaacgtgcgtgagagtaactgttcacgcagtgacggtatccaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaaccttagagataaggcgttcccttcggggacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcaagctcgcgagagtaagctaatctcttaaagccgttctcagttcggactgtaggctgcaactcgcctacacgaagtcggaatcgctagtaatcgcggatcagcatgccgcggtgaatacgttcccgggccttgtacacaccgcccgtcacaccatgggagtttgtaacgcccaaagtcggtggcctaacctttatggagggagccgcctaaggcgggacagatgactggggtgaagtcgtacagggggaaacccgt

SEQ ID NO: 4 - DNA, 1458 bp, Lacticaseibacillus rhamnosus
  misc_feature 1..1458: note = Lacticaseibacillus rhamnosus
  source 1..1458: mol_type = genomic DNA
tctgattattgaaaggtgcttgcttcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttccccttcgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaac